(19) United States
(12) Patent Application Publication
(10) Pub. No.: US 2012/0172804 A1
(43) Pub. Date: Jul. 5, 2012

(54) MEDICAMENT DELIVERY DEVICES

(75) Inventor: David Plumptre, Worcestershire (GB)

(73) Assignee: SANOFI-AVENTIS DEUTSCHLAND GMBH, Frankfurt am Main (DE)

(21) Appl. No.: 13/254,614

(22) PCT Filed: Mar. 24, 2010
(86) PCT No.: PCT/EP10/53814

§ 371 Date: Mar. 16, 2012

(30) Foreign Application Priority Data
Mar. 31, 2009 (EP) 09004647.5

(51) Int. Cl. A61M 5/315; A61M 5/20

(52) U.S. Cl. 604/154; 604/228

(57) ABSTRACT

A medicament delivery device (1) comprises a housing (2) for holding a medicament cartridge (9), a drive (18, 32) and a drive control means (46, 52). The medicament cartridge (9) has a medicament outlet and a bung (20) able to be driven via a piston rod (12, 14) driven by the drive force of the drive (18, 32) and controlled by the drive control means (46, 52). Additionally, the medicament delivery device (1) comprises a restraining means (38) for applying a restraining force to the piston rod (12, 14), in a direction opposite to the drive force. By varying the restraining force by the drive control means (46, 52) the movement of the bung (20) along the medicament cartridge can be controlled.

The present invention relates to improvements for a medicament delivery device, and in particular to improvements in a drive mechanism for a portable injection device for dispensing controlled quantities of a medicament.

Injection devices are known for the self-administration of a medicament by patients. For example, those suffering from diabetes may require regular injections of insulin; others may require regular injections of a growth hormone. Injection devices allow the patient to select a dose and to administer that dose. It is known to automate this process so that a user need only press a button and the injection device will dispense a selected dose of medicament. This relieves the patient of the task of controlling the amount dispensed while manually expelling the medicament from the injection device. This is a particular problem for the elderly, the infirm, those suffering from vision difficulties and those suffering from diabetes related problems that impair their faculties.

The medicament is typically contained within a cartridge located within the injection device. The cartridge has a bung or piston at one end, which is driven towards a second end of the cartridge to expel the medicament from the injection device. It is a problem that injection devices should be small enough to fit into a jacket pocket or a handbag without difficulty. At the same time, the injection device must be of a size that enables a piston or the like used to drive the cartridge bung within the cartridge to be moved both to a maximum dispense position within the cartridge and to be fully withdrawn from the cartridge to allow for replacement of the cartridge.

U.S. Pat. No. 7,125,395 describes a device for controlling delivery of an injectable fluid with the aim of providing a simpler hand-operable mechanism. A drive wheel is driven by the restoring force of a spiral spring. The drive is applied to one end of a flexible piston rod, the other end thereof contacting a bung of a medicament cartridge. The release of energy stored in the spiral spring, and so drive of the bung, is controlled by a rocking lever that engages with the drive wheel. The flexible piston rod allows the one end of the piston rod to be displaced relative to the axis of the medicament cartridge.

WO 02/28455 describes a medication delivery device aimed at combining compactness with improved accuracy. The device has a piston rod in the form of a tape bent over an arcuate guide, one end of the piston rod contacting a bung of a medicament cartridge. A motorised drive pushes the piston rod at a point along its length so that a drive force can be transferred to the bung along part of the length of the piston rod.

It is a disadvantage that these devices require a motor that can provide a significant drive torque to drive the bung and thereafter retract the piston through drive of the piston rod. This in turn requires more battery power leading to a larger form factor.

It is an aim of the present invention to alleviate the aforementioned disadvantages.

According to a first aspect of the present invention, there is provided a medicament delivery device comprising: a housing for holding a medicament cartridge, the medicament cartridge having a medicament outlet and a bung movable axially along the cartridge; a drive for applying a drive force to the bung via a piston rod; and a drive control means for controlling application of the drive force to the bung; characterized in that the medicament delivery device comprises restraining means for applying a restraint to the piston rod wherein the drive control means is operative to control the restraining means to release the piston rod to move under the influence of the drive for moving the bung along the cartridge.

In a preferred embodiment, the drive may comprise a resilient bias means such as a spring arrangement for biasing the piston rod against the bung. In this embodiment, the drive force is operative to push the bung axially along the medicament cartridge in order to dispense medicament from the outlet. The restraining means comprises an elongate element attached at one end thereof to the piston rod and operative to pull the piston rod against the force of the drive. The elongate element is preferably flexible but longitudinally non-extendible. Drive of the bung or retraction of the piston rod from the medicament cartridge is thereby effected in embodiments of the present invention by way of a ‘push-pull’ operation between the bias action of the spring arrangement and the oppositely directed force exerted by the elongate element.

The elongate element of the restraining means may be coupled to a mechanical and/or electro-mechanical actuator of the drive control means for facilitating controlled movement thereof under the bias action of the resilient bias means. Energy is stored in the resilient bias means, for example, by compression of a compression spring, and is released in a controlled and incremental way by the actuator to provide the drive for the piston rod, thereby to effect delivery of the medicament during use of the device. In other words, release of the restraint on the piston rod allows the elongate element to move or ‘pay-out’ whereby the resilient bias means can drive the piston rod. The electro-mechanical actuator may comprise a stepper or d.c. motor coupled to the elongate element via a first gear such that the elongate element is incrementally moveable by predetermined amounts under the action of the compression spring bias force. This permits a corresponding movement of the piston rod, and in turn the bung, to effect delivery of the medicament.

The medicament delivery device may include a user-operated mechanical actuator for retracting the piston of the medicament cartridge when empty thereby facilitating re-use of the device. Retraction of the piston by the user-operated actuator may also serve to ‘recharge’ the energy stored in the resilient bias means by, for example, compressing the compression spring. The user-operated actuator transfers energy from the user to the resilient bias means in a two stage movement, a first stage thereof facilitating removal of an empty medicament cartridge and a second stage thereof setting the device for re-use following insertion of a replacement cartridge. In this case, the end of the elongate element may be coupled to the elongate element via a second gear so that operation of the user-operated mechanical actuator retracts the piston rod against the bias force of the resilient bias means. The first and second gears may be combined in a gearing assembly. In an alternative embodiment, the user-operated mechanical actuator may be substituted by an electro-mechanical drive for retracting the piston and recharging the resilient bias means. In this case, the stepper or d.c. motor may be used to drive the piston rod into a retracted position such as to simultaneously compress the compression spring of the resilient bias means. The drive control means may include an electronic control for controlling movement of the elongate element by the stepper or d.c. motor. The user-operated actuator may include a holder for the medicament cartridge whereby retraction of the piston by the user-operated actuator simultaneously releases the cartridge from the housing, thereby facilitating replacement of the cartridge

The piston rod may be telescopically expandable for driving the bung and be telescopically collapsible for facilitating replacement of the cartridge. This provides for a reduction in the overall size of the medicament delivery device.

The elongate element may be helically wound about itself as the piston rod moves into a retracted condition. This flexibility allows the elongate element to pass over a surface such as a pulley wheel such that the gear and ratchet arrangement can be situated alongside the medicament container housing. Consequently, the restraining means does not need to be positioned to be axially coincident with the cartridge. This further provides for a reduction in the size of the medicament delivery device.

The elongate element may consist of a flexible belt or cable attached to the piston rod and the drive control means may be in the form of a braking means adapted to selectively pay out the belt or cable to allow the piston rod to move in the axial direction of the cartridge under the bias of the spring drive to drive the bung thereby dispensing medicament. Provision of the flexible belt or cable has the advantage that it provides for a reduction in the length of the device.

Preferably, the braking means comprises an electric motor adapted to selectively pay out the belt or cable. Where the braking mechanism also comprises a drive member in driving engagement with the belt, movement of the drive member may be controlled by the electric motor in order to selectively pay out the belt. Preferably, the electric motor controls the movement of the drive member by means of a rocker or ratchet device. In one embodiment, the motor is adapted to re-wind the belt or cable so as to move the piston in a second axial direction opposite to the first to re-set the drive mechanism. Alternatively, the belt or cable can be re-wound manually to re-set the drive mechanism.

Preferably, the spring arrangement comprises at least one helical compression spring. In particular, the resilient means may comprise two helical compression springs, in which case, a first helical compression spring may act between a housing member and a coupling such as a spring collar or sleeve and a second helical compression spring may act between the coupling and the piston to bias the piston in the axial direction to drive the bung. In a preferred embodiment, the piston has an axially extending piston body, which is telescopically received within the spring collar whilst the spring collar may be receivable within the housing member.

According to the present invention, there is further provided a medicament delivery device comprising: a housing for holding a medicament cartridge, the medicament cartridge having a medicament outlet and a bung movable axially along the cartridge; a drive for applying a drive force to the bung via a piston rod; and a drive control means for controlling application of the drive force to the bung; characterized in that the medicament delivery device comprises a user-operated actuator for retracting the piston rod to facilitate replacement of the medicament cartridge, and for simultaneously transferring energy from the user to the drive which is formed from a resilient bias means. In a preferred embodiment of this further aspect, the retraction of the piston rod against the drive transfers energy from the user to the resilient bias means in a two-stage movement, a first stage thereof facilitating removal of an empty medicament cartridge and a second stage thereof setting the device for re-use following insertion of a replacement cartridge.

Embodiments of the aspects of the invention defined above may be deployed in an injector pen, auto-injector or infusion device. Use of the resilient bias means provides for energy storage in the device which facilitates drive of the piston rod. This has the advantage of reducing reliance on the motor for driving the piston rod leading to a reduction in motor size and power requirements. The size of the motor and power requirement can be reduced to power the gearing and ratchet, the energy stored in the resilient bias means providing for the extension of the piston rod and consequential paying out of the elongate member.

A sensor may be provided for detecting movement of the elongate element. Embodiments of the present invention may be deployed in an auto-injector which may be of the pen-injector type.

The invention will now be further described, by way of example, with reference to the accompanying drawings in which like reference numerals designate like elements:—

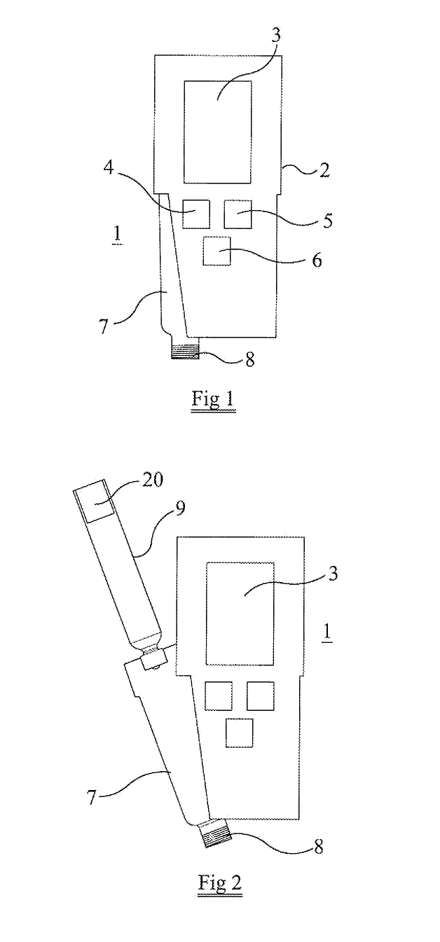

FIG. 1 is a front view of a medicament delivery device that may include an embodiment of the present invention;

FIG. 2 is a front view of the medicament delivery device of FIG. 1 with a medicament cartridge door shown in an open position for receiving a medicament cartridge;

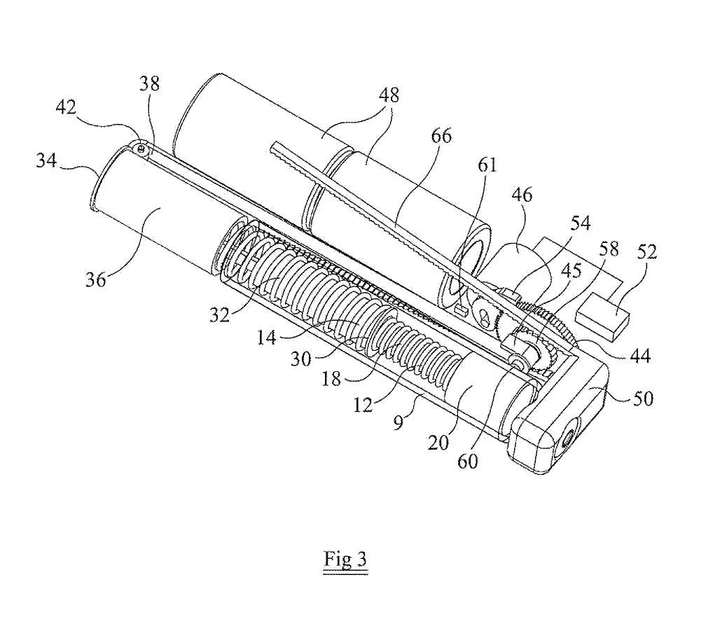

FIG. 3 is a perspective view of the principal components of a medicament delivery device embodying the present invention, showing a telescopic piston rod in an extended position;

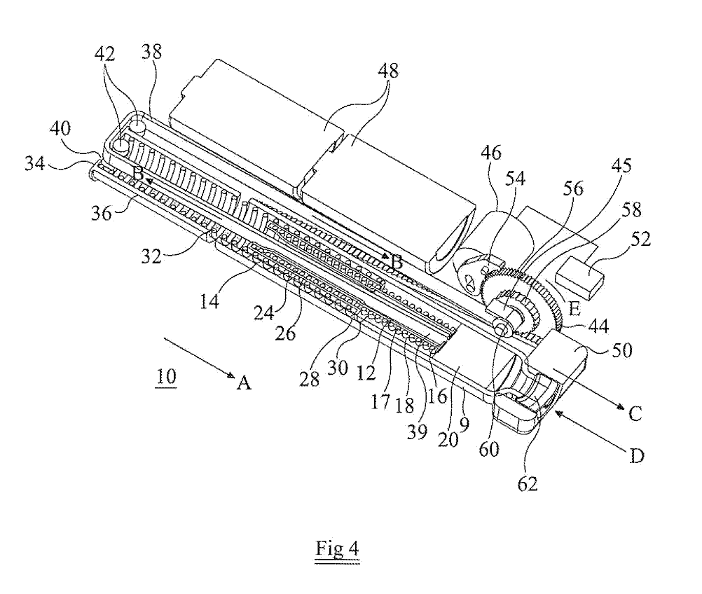

FIG. 4 is a sectional perspective view of the device of FIG. 3;

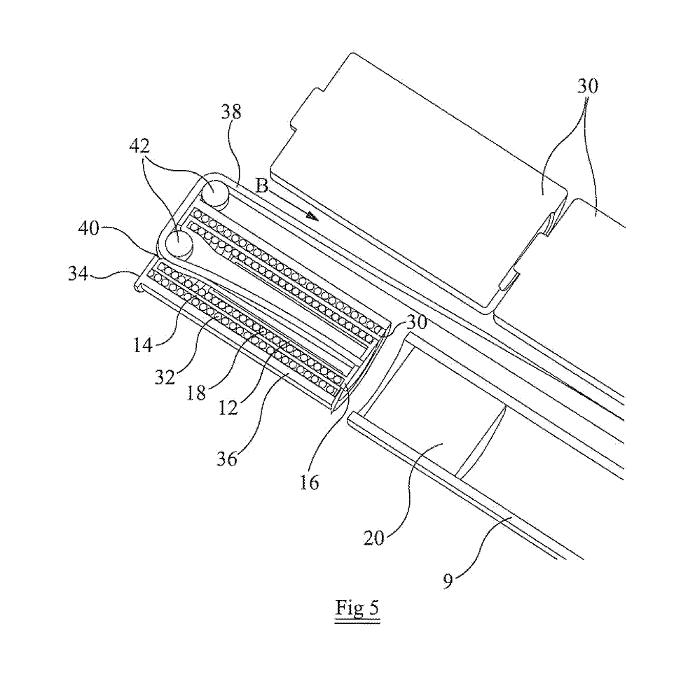

FIG. 5 is a part-sectional view of the device of FIGS. 3 and 4 showing the telescopic piston rod assembly in a retracted position;

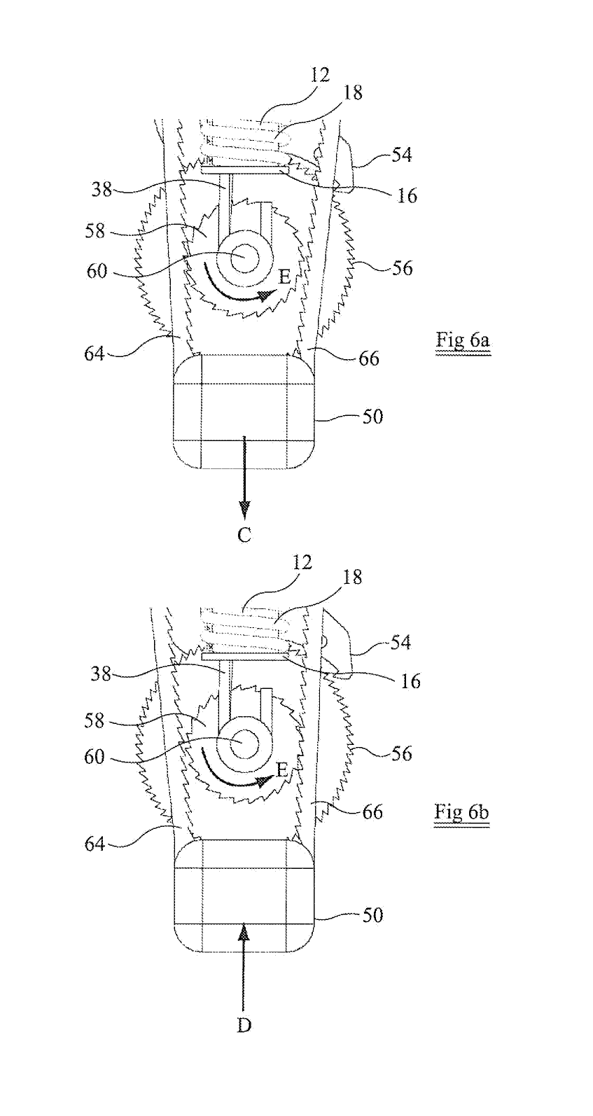

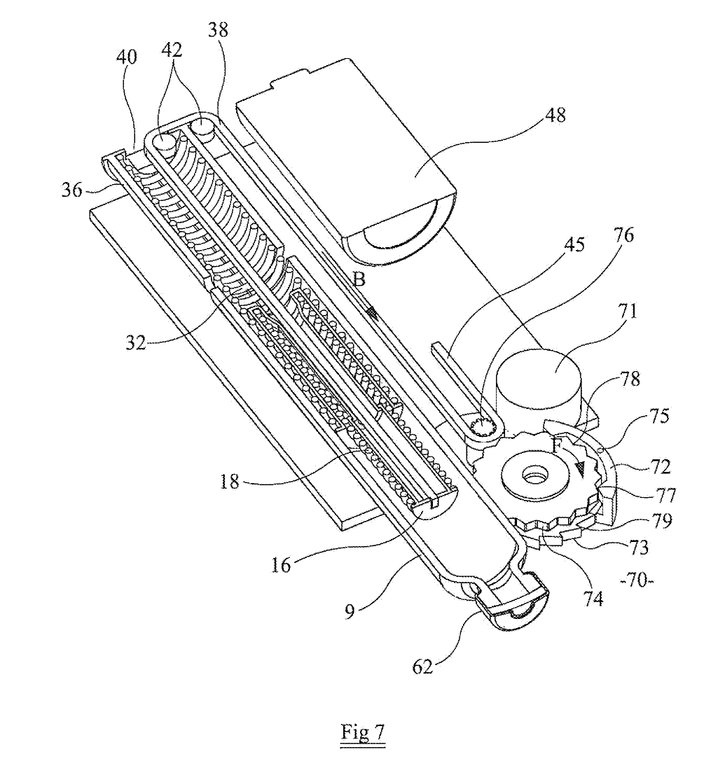

FIG. 6a shows a detail of a first gearing arrangement and one engagement with a user-operated actuator;

FIG. 6b shows a detail of the first gearing arrangement and another engagement with a user-operated actuator; and

FIG. 7 is a sectional view of an embodiment of the present invention showing the telescopic piston rod assembly with a modified gearing arrangement.

In FIG. 1, a medicament delivery device 1 comprises a case 2 having a display 3 for displaying functional information relating to the operation of the medicament delivery device, including the set dose, number of doses remaining in the medicament cartridge. User interface buttons 4, 5 and 6 are provided to allow the user to operate the injector including priming, setting a dose, opening a medicament cartridge holder and door 7, and activating the dispensing of the set dose. A threaded needle attachment 8 is provided to which a needle can be attached for dose delivery and subsequently removed and discarded. A cover (not shown) may be provided to fit over the lower portion of the case 2 to assist in protect the device from the ingress of particles and fluid. FIG. 2 shows the medicament delivery device 1 with the cartridge holder and door 7 in an open position for receiving a replacement medicament cartridge 9.

FIG. 3 is a perspective view of the principal operational components of a medicament device 10 embodying the present invention that may be fixed to a chassis (not shown) within the case 1 of FIGS. 1 and 2. FIG. 3 will be described in conjunction with the sectional view of FIG. 4 and shows a piston rod of the device 10 in a fully extended state when the medicament cartridge 9 is empty. The device 10 comprises the piston rod having a plunger 12 telescopically coupled to a sleeve 14. The plunger 12, of generally cylindrical form, has an end plate 16 at one end thereof that extends radially outwardly to support one end 17 of a first compression spring 18 that extends along the exterior of the plunger. The end plate 16 is urged towards a bung 20 of the medicament cartridge 9 by the compression spring 18. The sleeve 14 is tubular and has a double wall that defines an annular pocket 24 for receiving the other end 26 of the first compression spring 18. The plunger 12 and the sleeve 14 have inter-engaging rims 28 that cooperate to prevent them from coming apart when in the fully extended position. The sleeve 14 has a radially extending flange 30 against which abuts one end of a second compression spring 32 that extends along the exterior of the sleeve 14. The first and the second compression springs 18, 32 together form a resilient bias means of the device 10 which, in the absence of an opposing force, is operative to apply a bias force in the direction of Arrow A, whereby the piston rod is biased into an extended position as illustrated in FIGS. 3 and 4. The extended position is reached when the bung 20 has traversed the axial length of the medicament cartridge 9 as the last medicament dose is delivered. The other end of the second compression spring 32 abuts against an end plate 34 of a housing 36 for accommodating the piston rod when the device is in a retracted state (see FIG. 5). To re-use the device, the piston rod must be retracted into the housing 36 as shown in FIG. 5 so that the medicament cartridge 9 can be replaced and the resilient bias means ‘recharged’ for biasing the bung 20 axially along the cartridge 9 for delivering the medicament contained therein. As apparent from FIG. 5, when the device 10 is in the retracted state the plunger 12 sits within the sleeve 14, which in turn sits within the housing 36. In this condition, the first and second compression springs 14, 32 are in a compressed or ‘charged’ state.

The device 10 includes a restraining means in the form of an elongate element 38 which may be formed from a flexible but non-extendible belt. The elongate element 38 passes through the tubular sleeve 14 and the plunger 12, one end 39 of which is attached to the end plate 16 of the plunger 12. The elongate element 38 passes axially through the second compression spring 32 and out through an opening 40 in the end plate 34. The elongate element 38 makes two 90 degree turns over a pair of pulleys 42 that are pivotally supported on the chassis of the case 2 so as to extend parallel to and alongside the outside of the housing 36 and the medicament cartridge 9 to engage with a gear drive 44 at its other end 45. Application of a pulling force on the elongate element 38 in the direction of an Arrow B serves to counter the extension force of the resilient bias means, that is, the compression springs 18, 32. The pulling force may be equal and opposite to the extension force so the piston rod is stationary. The elongate element 38 is held in a stationary position by a braking device, lock or ratchet device as will be described in more detail below. The elongate member 38 may also be pulled against the extension force of the resilient bias means to retract the piston rod manually by the user and/or by a motorised drive 46 via the gear drive 44, as will be described in more detail below. A battery pack 48, shown schematically, is provided for powering the motorised drive 46.

The motorised drive 46 may be a stepper or d.c. motor under the control of an electronic control or actuator 52. The motorised drive 46 is coupled to the gear drive 44 via an eccentrically mounted pawl 54 which engages with a toothed gear 56 of the gear drive 44 in a ratchet type operation. The gear drive 44 comprises an inner gear wheel 58 of lesser diameter to that of the toothed gear 56. The inner gear wheel 58 has a spigot 60 for engaging the elongate element 38. The extension force of the resilient bias means puts the elongate element 38 under tension. When the telescopic piston rod is in the extended position shown in FIG. 4, the end 45 of the elongate element 38 is in its most extended position such that its free end 45 is close to the gear drive 44. The elongate element 38 must remain in contact with the spigot 60. The spigot 60 is provided with teeth (not shown) that cooperate with corresponding teeth (not shown) on the elongate element 38 so that if the gear drive 44 is driven to rotate in the direction of Arrow E in FIG. 4, the elongate element 38 is pulled in the direction of the Arrow B against the extension force of the resilient bias means. A guide (not shown) may be provided to hold the elongate element 38 in contact with the spigot 60. The guide may also serve to direct the free end 45 of the elongate element 38 into a free space in the device as it returns to a retracted state. The free space runs alongside the battery 48. The free end 45 of the elongate member may be held under tension by a constant-force spring or coiled spring to assist in maintaining engagement of the toothed belt with the corresponding teeth of the spigot. A sensor 61 may be provided for detecting movement of the elongate element 38. In this case, the pawl 54 slides over the toothed gear 56. When no drive is applied to the gear drive 44, the pawl 54 catches the teeth of the toothed gear 56 to hold or lock rotation thereof. This prevents extension of the elongate element 38 and therefore prevents extension of the telescopic piston rod.

When the device is in medicament delivery mode, the plunger 12 is movable from a fully retracted state as illustrated in FIG. 5 to the extended state as illustrated in FIG. 4 by releasing engagement of the pawl 54 from the toothed gear 56 in a ratchet operation. Engagement between the eccentrically mounted pawl 54 and the toothed gear 56 is controllably released by the electronic control 52. The electronic control 52 is operative to control the motorised drive 46 to rotate such that the pawl 54 is released to permit rotation of the gear drive 44 by a predetermined number of toothed gear 56 teeth. This ‘pays-out’ the elongate element 38 under the extension force of the resilient bias means by an amount that corresponds to the desired medicament dose to be delivered from the device.

Instead of the toothed engagement between the elongate element 38 and the spigot 60, the end 45 of the elongate element 38 may be crimped to the spigot 60. In this case the elongate element 38 is helically wrapped (not shown) around the spigot 60 when the telescopic piston is in the retracted position of FIG. 5. The elongate element 38 unwinds as the gear drive 44 is allowed to rotate by the ratchet operation of the pawl 54 and toothed gear 56 as described above against the direction of the Arrow E under the extension force of the resilient bias means which pushes the bung 20 axially along the medicament cartridge 9 during dispensing of the medicament.

When the medicament cartridge 9 is empty, the telescopic piston rod needs to be retracted into the position shown in FIG. 5 in order to allow replacement of the cartridge 9. The motorised drive 46 may be operative for electro-mechanically driving the gear drive 44 to rotate in the direction of the Arrow E in FIG. 4 to pull the elongate element 38 against the resilient bias means, and hence bring the bung 20 into the retracted position shown in FIG. 5. To conserve battery power, this operation may be carried out under a programmed control by the electronic control 52, slowly over a relatively long period of time. FIG. 5 shows part of a new medicament cartridge 9. However, in the illustrated embodiment, the device 10 is provided with a user-operable actuator 50 having a grip so that a patient can reset the device by manually retracting the piston rod into the housing 36 in order to replace the medicament cartridge 9. This manual operation also serves to recharge the energy stored in the resilient bias means. The user-operable actuator 50 has an aperture 62 for holding a neck of the medicament cartridge 9. To manually recharge the device 10, the patient grips the user-operable actuator 50 and pulls it in the direction of Arrow C in FIGS. 4 and 6a. The user-operable actuator 50 is provided with first and second rack members 64 and 66 respectively. The rack members 64, 66 are provided with teeth that engage with the teeth of the inner gear wheel 58. The spacing between the rack members 64 and 66 is preferably less than the diameter of the inner gear wheel 58. Initially, the teeth of the rack member 64 engage with the inner gear wheel 58 when the user-operable actuator 50 is pulled out of the device in the direction of the Arrow C as shown in FIG. 6a. During this action, the teeth of the rack member 66 slide over the corresponding teeth of the inner gear wheel 58, again see FIG. 6a. This action rotates the inner gear wheel 58 in the direction of the Arrow E shown in FIGS. 4, 6a and 6b which rotates the spigot 60 to impart a tensile force (i.e. pulling force) to the elongate member 38 as described above. This pulling action is transmitted to the end plate 16 of the plunger 12 by the elongate element 38 such as to overcome the extension force exerted by the compression springs 18 and 32. This in turn draws the piston rod towards the housing 36. As the user-operable actuator 50 is pulled out to its fullest extent, at which point the piston rod is retracted substantially half way along the medicament cartridge 9, the user-operable actuator 50 is pushed back into the device in the direction of the Arrow D of FIG. 6b. The rack 66 then engages with the inner gear wheel 58 and the teeth of the rack 64 slide out of engagement with it as shown in FIG. 6b. Consequently, the user can retract the piston rod the rest of the way back to the fully retracted state by pushing the user-operable actuator 50 back into the device 10 in the direction of the arrow D. During this operation, the pawl 54 slides over the teeth of the toothed gear 56. Once the user-operated actuator 50 has been pulled out to its fullest extent, the empty medicament cartridge 9 can be removed from the device 10 and replaced with one full of medicament. Pushing the user-operated actuator 50 back into the device completes the resetting of the resilient bias means. The above described mechanism provides for user reset of the device by the simple act of replacing the medicament cartridge 9. In other words, the device automatically recharges the drive during cartridge replacement.

The reset device 10 is ready to deliver metered doses of medicament. As apparent from the description above, when the device is in the reset state, the compression springs 18, 32 exert an extension force on the telescopic piston. This extension force is balanced or ‘restrained’ by the elongate element 38. The elongate element 38 is held in position by virtue of the pawl 54 being biased into locking engaging with the gear wheel 56. As the electronic control 52 drives the motorised drive 46, the eccentric drive allows the pawl 54 to lift over a predetermined number of teeth of the gear wheel 56 which in turn allows the gear drive 44 to rotate by a corresponding amount. This permits the elongate element 38 to move, with a corresponding extension of the telescopic piston rod, under the extension force of the compression springs 18, 32. The plunger 12 of the piston rod therefore pushes the bung axially along the medicament cartridge 9 to dispense a desired quantity of medicament from the device. In an alternative embodiment, a user operated ‘clicker’ may be adopted for releasing the pawl 54 over a predetermined number of teeth of the gear wheel 56 to deliver the desired dose of medicament.

FIG. 7 shows an alternative embodiment similar to the one described with reference to FIGS. 1 to 6 except that a different gearing 70 and motor 71 arrangement is adopted. This gearing arrangement 70 has a rocker 72 instead of the pawl 54 for controlling rotation of the gearing arrangement 70. The gearing arrangement 70 comprises a ratchet gear 73 and rocker gear 74 for driving the elongate element 38 is a similar manner to the first embodiment. In this embodiment, the rocker gear 74 is coupled to the ratchet gear 73 by a one-way ratchet (not shown) and may be driven by the motor 71 either directly or via intermediate gears (not shown).

The rocker gear 74 may be driven in a clockwise direction (direction of arrow F) by the motor 71 such that the ratchet gear 73 is also rotated in a clockwise direction due to the one-way ratchet coupling between the gears 73, 74. Rotation of the ratchet gear 73 causes a spigot gear 76 (analogous to spigot 60) to rotate anti-clockwise in a direction counter to arrow F. This movement of the spigot gear 76 pulls the elongate element 38 in the direction of arrow B, due to the engagement between the elongate element 38 and the spigot 76. Due to the engagement between the elongate element 38 and the piston rod, the piston rod is retracted, allowing access to the medicament cartridge 9. It will be appreciated that the engagement between the elongate element and the spigot gear 76, and the retraction of the piston rod by the elongate element is analogous to the previous embodiment.

When the rocker gear 74 is driven in an anti-clockwise direction (opposite to arrow F) by the motor 71 the ratchet gear 73 is not rotated due to the one-way ratchet coupling. In this case, rotation of the rocker gear 74 causes the teeth 77 of the rocker 72 to disengage with the corresponding grooves 78 of the rocker gear 74. This action causes the rocker 72 to rock about its central axis 75, and the rocker teeth 77 to disengage with the grooves 79 on the ratchet gear 73. As the ratchet gear 73 disengages with the rocker 72, the ratchet gear 73 slips in the direction counter to arrow F due to the tension exerted by the compression springs 18, 32 and the engagement between the piston rod, elongate element 38 and spigot gear 76 until the teeth 77 of the rocker 72 re-engage with the grooves 79 of the ratchet gear 73. This action ‘pays-out’ the elongate element 38 under the extension force of the resilient bias means 18, 32 by an incremental amount and consequently, as the motor continues to rotate, delivers the desired medicament dose from the device in a manner similar to the first embodiment.

Whereas the invention has been described in relation to what is presently considered to be the most practical and preferred embodiments, it is to be understood that the invention is not limited to the disclosed arrangements but rather is intended to cover various modifications and equivalent constructions included within the scope of the invention. For example the restraining means need not comprise a toothed belt but could comprise a cable or other flexible member connected to the piston rod and a suitable braking device to allow controlled advancement of the piston rod.

1. A medicament delivery device comprising:
a housing configured to hold a medicament cartridge, the medicament cartridge having a medicament outlet and a bung movable axially along the cartridge; a drive configured to apply a drive force to the bung via a piston rod; and
a drive control means configured to control application of the drive force to the bung; characterized in that the medicament delivery device comprises restraining means configured to apply a restraint to the piston rod, wherein the drive control means is operative to control the restraining means to release the piston rod to move under the influence of the drive for moving the bung along the cartridge.

2. A medicament delivery device according to claim 1, wherein the restraining means comprises an elongate element attached at one end thereof to the piston rod and is operative to pull the piston rod against the force of the drive.

3. A medicament delivery device according to claim 2, wherein the drive comprises a resilient bias means configured to bias the piston rod against the bung for pushing the bung along the medicament cartridge in order to dispense medicament from the outlet.

4. A medicament delivery device according to claim 3, wherein the restraining means is coupled to an actuator configured to facilitate controlled movement thereof under the bias action of the resilient bias means, thereby controllably releasing energy stored therein to drive the piston rod, and in turn the bung, to effect delivery of the medicament during use of the device.

5. A medicament delivery device according to claim 4, wherein the actuator comprises a motor such that the elongate element is incrementally moveable by predetermined amounts against the action of the resilient bias means.

6. A medicament delivery device according to claim 3, comprising a user-operated actuator for retracting the piston rod to facilitate replacement of the medicament cartridge, and for simultaneously transferring energy from a user to the resilient bias means.

7. A medicament delivery device according to claim 6, wherein retraction of the piston rod against the drive transfers energy from the user to the resilient bias means in a two-stage movement, a first stage thereof facilitating removal of an empty medicament cartridge and a second stage thereof setting the device for re-use following insertion of a replacement cartridge.

8. A medicament delivery device according to claim 6, wherein the elongate element is coupled to the user-operated actuator by way of a first gear so that operation of the user-operated actuator by the user retracts the piston rod against the drive.

9. A medicament delivery device according to claim 5, wherein the motor is coupled to the elongate element by way of a second gear for facilitating the incremental movement.

10. A medicament delivery device according to claim 8, wherein the first and/or second gears include a ratchet device.

11. A medicament delivery device according to claim 2, wherein the elongate element is flexible but longitudinally non-extendible.

12. A medicament delivery device according to claim 11, wherein the elongate element passes over a surface for changing the path thereof so that the actuator can be situated alongside the medicament container housing.

13. A medicament delivery device according to claim 3, wherein the resilient bias means comprises one or more compression springs.

14. A medicament delivery device according to claim 1, wherein a sensor is provided for detecting movement of the elongate element.

15. A medicament delivery device according to claim 1 wherein the piston rod is telescopically expandable for driving the bung and is telescopically collapsible for facilitating replacement of the cartridge.

16. A medicament delivery device according to claim 15, wherein the piston rod comprises a piston body telescopically receivable within a coupling, and the resilient bias means comprises a first compression spring acting between the coupling and the piston body, and a second compression spring acting between a housing member and the coupling to bias the piston body in the axial direction of the medicament cartridge to drive the bung.

17. A medicament delivery device according to claim 16, wherein the piston body and the coupling are telescopically receivable within the housing member.

18. A medicament delivery device according to claim 6, wherein the user-operated actuator includes a holder for the medicament cartridge.
